(19) United States
(12) Patent Application Publication
(10) Pub. No.: US 2019/0298330 A1
(43) Pub. Date: Oct. 3, 2019

(54) Pterygium Lid Speculum

(71) Applicant: ALBERTO MARTINEZ, Rockville, MD (US)

(72) Inventor: ALBERTO MARTINEZ, Rockville, MD (US)

(21) Appl. No.: 16/373,547

(22) Filed: Apr. 2, 2019

Related U.S. Application Data
(60) Provisional application No. 62/651,309, filed on Apr. 2, 2018.

(51) Int. Cl. A61B 17/02; A61B 1/32

(52) U.S. Cl. CPC A61B 17/0231; A61B 17/0206; A61B 1/32

(57) ABSTRACT

A lid speculum apparatus is described. The speculum operates in an anatomically correct manner to provide full exposure of the eye during pterygium surgery. The speculum has a first blade and a second blade of the same size that are long enough to support the entire longitude of the upper and lower lids. With such an arrangement, the blades will provide full exposure of the globe including the nasal and temporal areas. The speculum is configured to open and close with a screw-based mechanism, and is designed to remain in position without adjustment during use. The blades are preferably curved to provide less pressure points than conventional straight blades.

### CONTINUITY

This application is a non-provisional patent application of provisional patent application No. 62/651,309, filed on Apr. 2, 2018, and priority is claimed thereto.

### FIELD OF THE PRESENT INVENTION

The present invention relates to the field surgical tools, and more specifically relates to an eyelid speculum configured to retract the eyelids of an eye of a patient comfortably and effectively for pterygium surgery.

### BACKGROUND OF THE PRESENT INVENTION

Lid speculums are used to retract an eyelid when performing an ophthalmic procedure. For example, the eyelids are retracted to perform pterygium surgery.

Unfortunately, conventional lid speculums of the prior art do not operate in an anatomically correct manner, which often causes patient discomfort. Additionally, such lid speculums of the prior art do not provide complete lid retraction on the nasal and temporal areas of the eye, which causes patient and surgeon discomfort.

Conventionally, an eyelid speculum has a pair of small blades that do not follow the full anatomical curvature of the eyelids. The blades maintain a constant two-dimensional plane of orientation as they are opened, pulling directly away from each other. When fully opened, the blades do not provide complete coverage and support to the nasal and temporal areas of the eyelids, causing the nasal and temporal areas of the eyelid to protrude out of the speculum and into the surgical field of the surgeon.

It is known that the vast majority of pterygium develops on the nasal side of the eye, but is not uncommon to develop a “second head” on the temporal side of the eye in the “pterygium belt” region located between 30° N and 30° S of the equator of the eye. Current lid speculum blades do not provide support to the entirety of the lids, which in some cases causes the temporal and nasal sides of the lids to protrude out of the speculum and cover some of the nasal and temporal areas of the eye. This represents a technical problem for the surgeon performing the pterygium surgery because it requires re-adjustment of the speculum in order to expose the areas on which the operation is performed.

Additionally, conventional lid speculums add strain, and produce kinked pressure points on the eyelids of the patient. Conventional lid speculums often require readjustment throughout the surgical process, unnecessarily prolonging the procedure. It would be desirable to develop a lid speculum that would operate in a more anatomically correct manner by providing better exposure of the nasal and temporal sides of the eye in order to help improve the outcomes of pterygium surgery.

Thus, there is a need for a new form of Ptery speculum equipped with long thin blades which are curved such that they follow the anatomical curvature of the upper and lower eyelids, and provides full support to the nasal and temporal areas of the eyelids. Such an apparatus is configured to completely expose the corners of the eye in order to increase patient and surgeon comfort during pterygiectomy. Additionally, such an apparatus facilitates full exposure in order to perform a single or double-headed pterygiectomy without the need of constantly re-adjusting the speculum, and without producing kinked pressure points on the eyelids which cause patient discomfort.

### SUMMARY OF THE PRESENT INVENTION

This Summary of the Invention is provided to introduce a selection of concepts in a simplified form that are further described below in the Detailed Description. This Summary is not intended to identify key features or essential features of the claimed subject matter, nor is it intended to be used to limit the scope of the claimed subject matter.

The present invention is a Ptery Speculum apparatus which is anatomically correct such that it facilitates comfort of the patient while simultaneously and effectively holding the eyelid at the ideal position for successful ptergiectomy or similar procedure. The present invention increases patient comfort by following the anatomical path of the eyelids of the patient, and reducing localized pressure points between the eyelids and the speculum.

Additionally, the present invention increases the comfort of the surgeon by providing better exposure of the areas on which the operation is performed. Similarly, use of the present invention decreases the overall surgery time and increases surgeon efficiency by eliminating the necessity of re-adjusting (switching) the speculum one or more times during surgery. The present invention is configured to cover all eyelashes of the patient, and is preferably composed of surgical grade stainless steel.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

The present invention will be better understood with reference to the appended drawing sheets, wherein:

FIG. 1 depicts a front view of a first embodiment of the pterygiectomy eyelid speculum of the present invention.

FIG. 2 exhibits a flow-chart detailing the process of use of the present invention.

### DETAILED DESCRIPTION OF THE PREFERRED EMBODIMENT

The present specification discloses one or more embodiments that incorporate the features of the invention. The disclosed embodiment(s) merely exemplify the invention. The scope of the invention is not limited to the disclosed embodiment(s). The invention is defined by the claims appended hereto.

References in the specification to “one embodiment,” “an embodiment,” “an example embodiment,” etc., indicate that the embodiment described may include a particular feature, structure, or characteristic, but every embodiment may not necessarily include the particular feature, structure or characteristic. Moreover, such phrases are not necessarily referring to the same embodiment, Further, when a particular feature, structure, or characteristic is described in connection with an embodiment, it is submitted that it is within the knowledge of one skilled in the art to effect such feature, structure, or characteristic in connection with other embodiments whether or not explicitly described.

The present invention is a speculum apparatus for use on patients by a surgeon performing a pterygiectomy. The present invention is equipped with a first blade (10) and a second blade (20). The first blade (10) is disposed in communication with a first inner linkage arm (30), and the second blade (20) is disposed in communication with a second inner linkage arm (40). The first inner linkage arm (30) and the second inner linkage arm (40) intersect at a central pivot point (50). The central pivot point (50) is preferably equipped with a lead screw (60), which provides tension to the central pivot point (50), ensuring that the first inner linkage arm (30) and second inner linkage arm (40) maintain a consistent position after the speculum is set at the ideal location and angle.

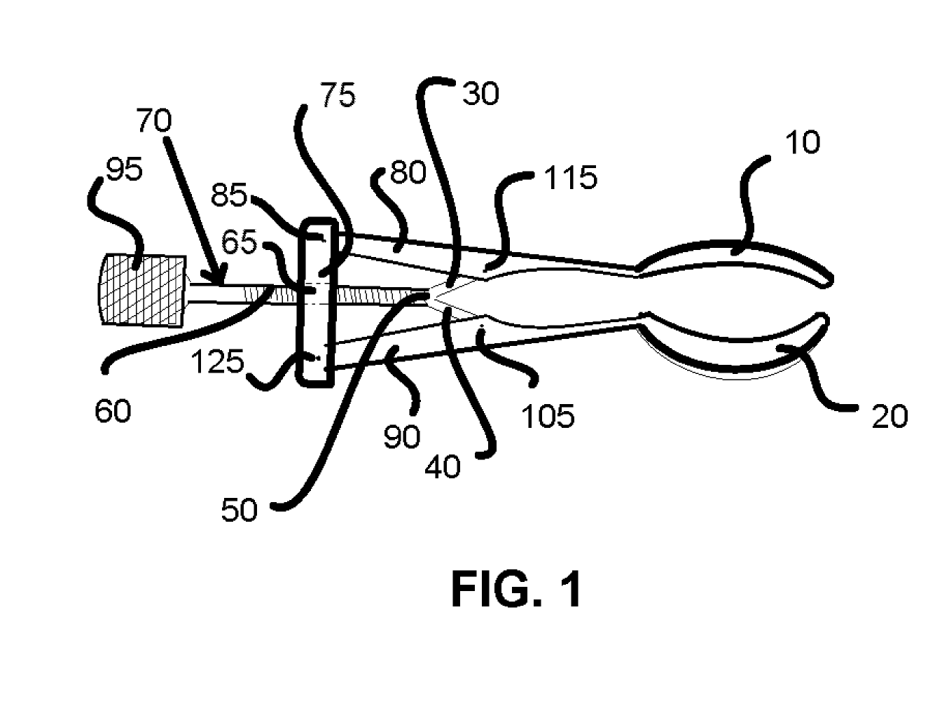

It should be understood that, when in use, the first inner linkage arm (30) and second inner linkage arm (40) are disposed on the temporal side of the present invention. The first inner linkage arm (30) and second inner linkage arm (40) are configured to move the first blade (10) ad second blade (20) via a manipulator (70) disposed in communication with a first outer linkage arm (80) and a second outer linkage arm (90). The first inner linkage arm (30) is in communication with the first outer linkage arm (80) via a first outer pivot point (115). The second inner linkage arm (40) is in communication with the second outer linkage arm (90) via a second outer pivot point (105), as shown in FIG. 1. Additionally, some embodiments of the present invention may be outfitted with one or more handles to facilitate maneuvering of the present invention into the ideal position for use.

The lead screw (60) of the manipulator (70) is configured to turn through a threaded aperture (65) of a base bar (75) of the apparatus. The lead screw (60) is preferably pivotally attached to the first inner linkage arm (30) and second inner linkage arm (40). In preferred embodiments, the present invention is equipped with a first outer linkage arm (80) and a second outer linkage arm (90) which are pivotally connected to the first inner linkage arm (30) and the second inner linkage arm (40) respectively. The first outer linkage arm (80) and second outer linkage arm (90) extend and are connected to the blades (10, 20) of the speculum. The first outer linkage arm (80) is also in communication with a first base pivot point (85), and the second outer linkage arm (90) is in communication with a second base pivot point (125) as shown in FIG. 1.

The lead screw (60) is attached to a head (95) of the manipulator (70) that can be rotated by a surgeon. Rotating the head (95) of the manipulator (70) translates the rotation to the lead screw (60) moving the arms to either retract or close the blades (first blade (10) and second blade (20)). The head (95) is preferably equipped with a textured grip to facilitate hand rotation of the manipulator (70) via the head (95) of the lead screw (60). Although a manually actuated lead screw (60) is shown and described in FIG. 1, it is to be understood that the lead screw (60) may be rotated automatically through a motor or other device.

The first blade (10) and second blade (20) of the apparatus are preferably curved similar to that of the curvature of an eyelid when opened as shown in FIG. 1. As such, the first blade (10) and second blade (20) are preferably thinner in width than that of conventional speculum blades, and the curvature is readily apparent. It is envisioned that the degree of curvature of the speculum apparatus of the present invention is preferably available in three sizes, namely a small size, a medium size, and a large size. As with the curvature, the length of the first blade (10) and second blade (20) of the present invention are preferably manufactured in multiple sizes to account for eyelids of varying widths. The first blade (10) is configured to retract a top eyelid of a patient, and the second blade (20) is configured to retract a bottom eyelid of the patient. Eyelashes of the patient are also restrained by the first blade (10) and second blade (20) of the present invention.

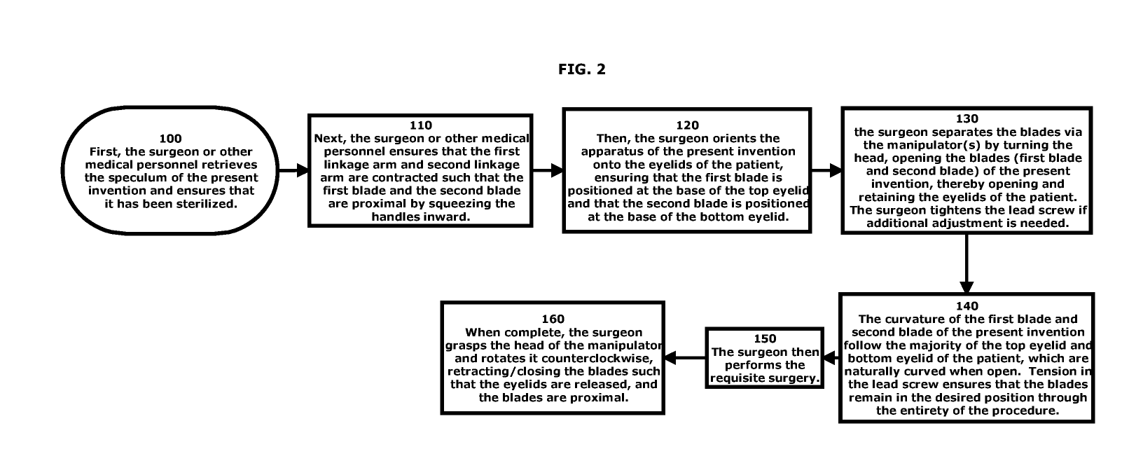

The process of use of the present invention, as depicted in FIG. 2, is preferably as follows:

1. First, the surgeon or other medical personnel retrieves the speculum of the present invention and ensures that it has been sterilized. (100)

2. Next, the surgeon or other medical personnel ensures that the first linkage arm and second linkage arm are contracted such that the first blade and the second blade are proximal by squeezing the handles inward. (110)

3. Then, the surgeon orients the apparatus of the present invention onto the eyelids of the patient, ensuring that the first blade is positioned at the base of the top eyelid and that the second blade is positioned at the base of the bottom eyelid. (120) The eyelashes of the patient are restricted via the first blade (10) and second blade (20) as well.

4. After the present invention is firmly oriented in the correct position, the surgeon separates the blades via the manipulator(s) by turning the head, opening the blades (first blade and second blade separating) of the present invention, thereby opening and retaining the eyelids of the patient. The surgeon tightens the lead screw if additional adjustment is needed. (130)

5. The curvature of the first blade and second blade of the present invention follow the majority of the top eyelid and bottom eyelid of the patient, which are naturally curved when open. Tension in the lead screw ensures that the blades remain in the desired position through the entirety of the procedure. (140)

6. The surgeon then performs the requisite surgery. (150)

7. When complete, the surgeon grasps the head of the manipulator of the present invention and rotates it counter-clockwise, retracting/closing the blades of the present invention such that the eyelids are released, and the blades are proximal. (160)

In short, it should be understood that the present invention is a lid speculum that operates in an anatomically correct manner and provides full exposure of the eye for pterygium surgery and other similar surgical procedures. The speculum has a first blade (10) and a second blade (20) that follow the entire anatomical path of the upper and lower eyelids. The present invention is constructed such that the first blade (10) and second blade (20) provide full support to the entire area of the upper and lower lids, unlike conventional speculums, which employ flat, lateral, or non-anatomically correct blades.

With such an arrangement, the temporal and nasal sides of the eye are fully exposed via the present invention, facilitating performance of a single or double headed pterygiectomy without requiring the surgeon to constantly re-adjust the speculum, and without producing kinked pressure points on the eyelids which cause patient discomfort.

The first blade (10) and the second blade (20) preferably each have a curved surface that allows the surgeon to expose most of the superior and inferior conjunctiva. The curved surface is more accommodative than straight speculum blades for pterygium surgery since a graft has to be collected from the superior conjunctiva area and the curved surface would also allow the surgeon to better visualize and operate on that area. Additionally, the curved blades provide less pressure points than straight blades.

Similarly, in some embodiments of the present invention, the first blade (10) and second blade (20) may be equipped with a second curved surface that conforms to the anatomical contour of the cornea. This secondary curved surface is preferably disposed on a base side of the first blade (10) and second blade (20), as opposed to the top side.

Embodiments of the present invention are preferably composed of surgical grade stainless steel, although other similar materials may be employed as long as they may be easily sterilized. While multiple sizes of the present invention are preferably envisioned, it should be generally noted that the small embodiment of the present invention has a chord length of 23 mm, and the large embodiment has a chord length of 28 mm. It should be understood that the present invention is not limited to these sizes, and that other chord lengths may be available. In some embodiments, an inner chord length may differ from that of an outer chord length.

Additionally, it should be understood that the present invention is suitable for use for other forms of eye surgery, including, but not limited to Pingueculectomy procedures.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I claim:

1. An eyelid speculum comprising:
a first inner linkage arm;
a second inner linkage arm;
a first outer linkage arm;
a second outer linkage arm;
a first blade, said first blade affixed to said first outer linkage arm;
a second blade, said second blade affixed to said second outer linkage arm;
a manipulator, said manipulator in communication with said first inner linkage arm and said second inner linkage arm;
wherein said first inner linkage arm is in communication with said first outer linkage arm via a first outer pivot point;
wherein said second inner linkage arm is in communication with said second outer linkage arm via a second outer pivot point;
a base bar, said base bar equipped with a threaded aperture;
a lead screw, said lead screw in communication with said manipulator, said first inner linkage arm, and said second inner linkage arm; and
wherein said lead screw is screwed into said threaded aperture, centered between said first outer linkage arm and said second outer linkage arm.

2. The apparatus of claim 1, further comprising: a head, said head disposed on an end of said manipulator; and
wherein said head is textured.

3. The apparatus of claim 1, wherein said first blade and said second blade are curved; and
wherein the curvature of said first blade and said second blade is anatomically correct.

4. The apparatus of claim 1, wherein said first blade is configured to retract and restrain a top eyelid when actuated via the manipulator; and
wherein said second blade is configured to retract and restrain a bottom eyelid when actuated via the manipulator.
